(19) United States
(12) Patent Application Publication
(10) Pub. No.: US 2012/0215293 A1
(43) Pub. Date: Aug. 23, 2012

(54) HIGH COMFORT HOT OR COLD THERMAL THERAPY PAD

(76) Inventor: Clinton O. Fruitman, Chandler, AZ (US)

(21) Appl. No.: 13/400,299

(22) Filed: Feb. 20, 2012

Related U.S. Application Data
(63) Continuation-in-part of application No. 11/025,615, filed on Dec. 29, 2004, now Pat. No. 8,121,679.

(51) Int. Cl. A61F 7/02

(52) U.S. Cl. 607/96

(57) ABSTRACT

A comfortably, yet highly efficient therapy pad that is effective in delivering cold or hot thermal therapy for the relief of pain, inflammation, swelling, and muscle stiffness.

### CROSS-REFERENCE TO RELATED APPLICATIONS

This application is a continuation-in-part of currently pending application having Ser. No. 11/025,615, Dec. 29, 2004 which is herein incorporated in its entirety.

### FIELD OF INVENTION

This invention relates, generally, to a method and apparatus for cold or heat therapy and the materials from which effective cold and heat therapy can be applied. More particularly, the present invention relates to a high thermal mass pad composed of a polymer matrix entrapping high specific heat fillers.

### BACKGROUND OF THE INVENTION

Traditional heat therapy may predate mankind as even animals are known to soak themselves in hot springs. Heat usually feels good because of the increased circulation it generates in affected tissue. This increased circulation relieves pain by improving tissue oxygenation and removing metabolic toxins. The disadvantage of heat therapy is that it can aggravate an inflammatory response. In addition, issues of over temperature and prolonged heating side effects are common.

Cold therapy is less often chosen by individuals for pain treatment because of the discomfort of ice on the skin. However, it is widely recognized as a powerful treatment for many types of pain by physicians and therapists and is often used for acute injuries. This therapy has three known mechanisms of action:
A. Although initially uncomfortable, cold contact is a heat sink that acts as a powerful anti-inflammatory by rapidly drawing heat out of affected tissue.
B. Cold temperatures have a direct anesthetic and numbing effect on sensory nerves.
C. Cold sensation on the skin can irritate A fibers under the skin which to some extent gates the deeper C fibers trying to convey pain impulses to the brain.

Drawbacks to traditional cold therapy are not limited to cold discomfort. With prolonged application and excessively reduced temperature, cold contact can cause decreased blood supply producing shivering, muscle tension, skin redness and even cold burns. To reduce risks and improve comfort, therapists often apply cold pads to the body through layers of cloth which unfortunately slows the action of the pads and reduces their clinical effectiveness.

With traditional heat therapy, the drawbacks include discomfort, burns, inflammatory aggravation and for the professional therapist, burn related liability expenses. Liability often minimizes a therapist's use of heat. To reduce risks and improve comfort, therapists often apply hot pads to the body through layers of cloth which also slows the action of the pads and reduces their clinical effectiveness.

A further drawback of conventional hot and cold therapy is economic in that the cycle lives of heating and chilling pads are short thereby requiring frequent costly replacements.

### SUMMARY OF THE INVENTION

In general, the present invention provides a hot and cold therapy device with a unique material that is simultaneously capable of safely, comfortably and rapidly delivering hot or cold thermal therapy in a highly effective fashion.

In one exemplary embodiment of the invention, the hot and cold therapy device includes a polymer matrix comprising a soft, solid, gel like elastomer with high specific heat and low thermal conductivity, and a filler combined with the polymer matrix wherein the filler comprises high specific heat and high thermal conductivity particles. The hot and cold therapy device of the present invention may be formed by casting or injection molding. This produces a thermal reservoir with high thermal mass and a slow but effective heat transfer rate. When applied directly to the skin, the soft conformable nature of the material combined with its slow heat transfer rate (which is governed by the insulating property of the polymer matrix and the conductive property of the filler) provides high levels of patient comfort even at temperatures between 20° F. and 135° F. without the typical hot or cold side effects.

In another exemplary embodiment of invention, the hot and cold therapy device comprises a solid thermal reservoir elastomer that is a 25% to 50% aluminum oxide filled gel like silicone with a combined Shore OO hardness of 10 to 25.

In still another exemplary embodiment of the invention, the hot and cold therapy device comprises a thermal reservoir where the elastomeric component of the thermal reservoir is comprised of an oil gel SEBS rubber with a combined Shore OO hardness of 10 to 25.

In yet another exemplary embodiment of the present invention, the shape of the therapy device is formed as a hollow shell with a filled polymer membrane of thickness between about 0.5 mm and 2 mm, and a cavity that can be filled with high specific heat materials which may include solids, liquids or gels.

In still another exemplary embodiment of the present invention, the hot and cold therapy device comprises a filled polymer layer of thickness between about 0.5 mm and 5 mm which is attached to an alternative heating or cooling device on at least one surface where therapeutic contact can be made.

### BRIEF DESCRIPTION OF THE DRAWINGS

FIG. 1 is a perspective view of an exemplary embodiment of the hot and cold therapy device of the present;

FIG. 2 is a perspective cut away view of another exemplary embodiment of the hot and cold therapy device of the present invention;

FIG. 3 is a perspective view of yet another exemplary embodiment of the hot and cold therapy device of the present invention.

FIG. 4 is a table showing relative thermal values for water, silicone rubber, and materials commonly used as thermal fillers;

FIG. 5 is a graph showing skin temperatures measured at time intervals for conventional ice packs, hydrogel pads, and two different sizes of the hot and cold thermal therapy pad of the present invention; and

FIG. 6 is a graph showing skin temperatures measured at time intervals for the hot and cold thermal therapy pad of the present invention and conventional wet silicone gel heating pads.

### DETAILED DESCRIPTION OF EXEMPLARY EMBODIMENTS

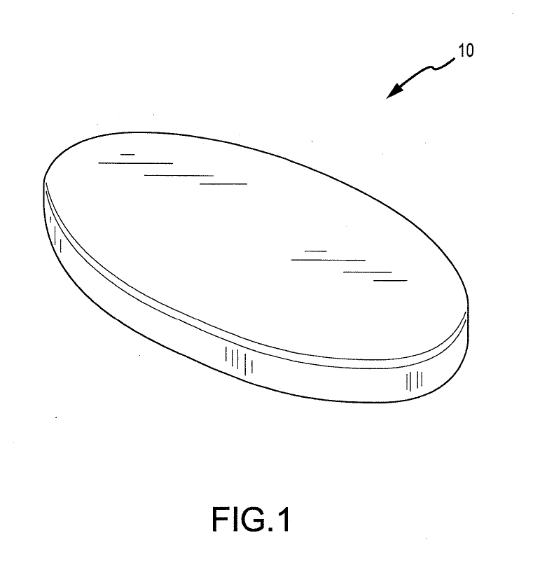

A perspective view of the hot and cold therapy device 10 of the present invention is shown in FIG. 1. Hot and cold therapy device 10 comprises a pad made of a polymeric matrix and a filler. The polymeric matrix material is combined with the filler material to form the pad. The material used for the polymeric matrix has low thermal conductivity and the material used for the filler has a high thermal conductivity. In addition, both the polymeric matrix material and the filler material may comprise materials having high specific heat.

For example the hot and cold therapy device 10 may comprise a polymeric matrix made of a polymer material having thermal conductivity between about 0.1 to 0.00001 Joules/cm sec. ° K and a filler made of a material having thermal conductivity between about 0.1 to 4 Joules/cm sec. ° K. In addition, both the polymer material and the filler material may have a specific heat of between about 300 to 4500 Joules/Kg·° K.

In another exemplary embodiment, the hot and cold therapy device 10 may be a pad comprising about 50 to 75% weight silicone rubber and about 25 to 50% weight aluminum oxide.

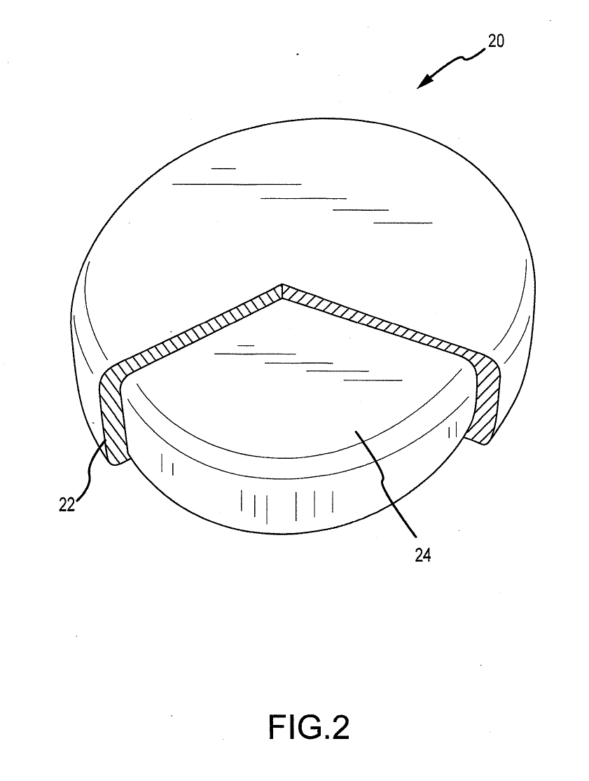

FIG. 2 is a perspective cut away view of another exemplary embodiment of the hot and cold therapy device 20 of the present invention having a hollow elastomer body 22 made of a polymeric matrix and a filler in accordance with the hot and cold therapy device 10 described above with reference to FIG. 1 and a material reservoir 24 which may comprise an alternative solid, liquid, or gel material. The hollow elastomer body 22 includes an opening in which the material reservoir 24 is contained.

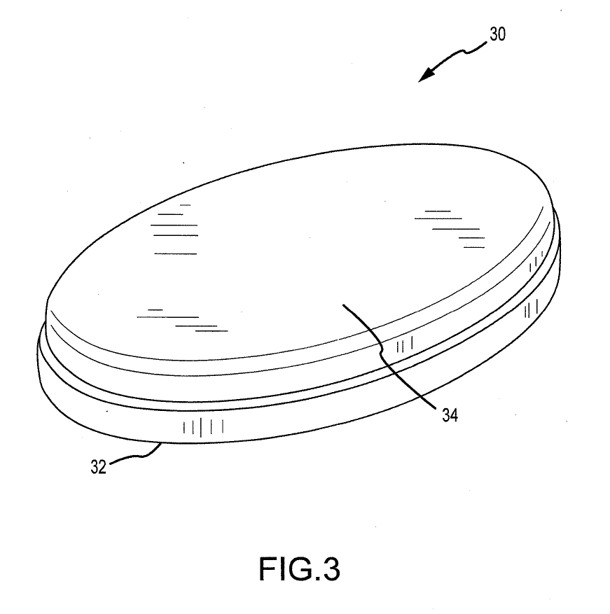

FIG. 3 is a perspective view of yet another exemplary embodiment of the hot and cold therapy device 30 of the present invention. Hot and cold therapy device 30 includes a polymer layer 32 made of a polymeric matrix and a filler in accordance with the hot and cold therapy device 10 described above with reference to FIG. 1 and an alternative heating and/or cooling device 34 attached to the polymer layer 32 so that the polymer layer 32 can be applied to or make contact with a user of the device 30.

### Fabrication

One familiar with the art will realize that this device can be fabricated in many different shapes, sizes, and contours and can have various physical properties according to the type of the polymer, the type and quantity of the filler, and the percentage of plasticizers used in the mixture.

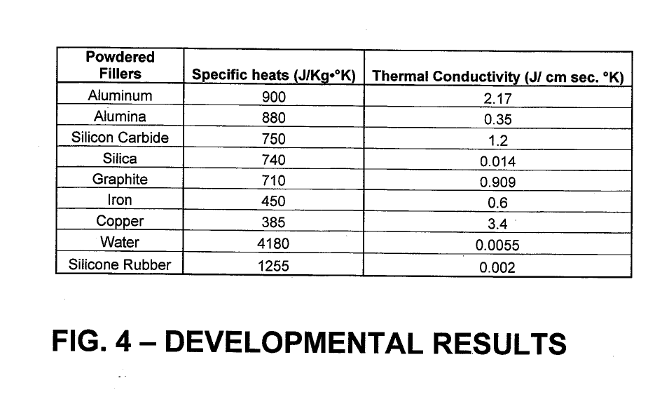

Many soft polymers can be effectively used in this invention. In preferred exemplary embodiments, the polymer should be very soft, for example in the range of about 10 to 30 on the Shore OO scale of hardness. Examples of super soft resins that can be effectively used in the present invention include, but are not limited to, Urethanes, Vinyls, Silicones, SEB and SEBS elastomers and oil gel dispersions of SEB and SEBS. Ideal embodiments employ fillers having good heat conductivity and high specific heat which are dispersed into the soft polymers of low thermal conductivity (See FIG. 4).

One preferred exemplary embodiment of the present invention is comprised of
(a) a soft polymer 50 wt. % to 75 wt. % Silicone Rubber with a standard Shore OO hardness of 30 or less, having high specific heat and low thermal conductivity combined with
(b) a 25 wt. % to 50 wt. % filler of 4 to 15 micron platy white aluminum oxide having high specific heat and high thermal conductivity.

FIG. 4 illustrates relative thermal values for water, silicone rubber, and materials commonly used as thermal fillers.

In both hot and cold therapy regimes, the present invention has been very effective in relieving various types of neuromuscular and skeletal pain, inflammation and swelling. In addition, patients have found it to be very comfortable to use.

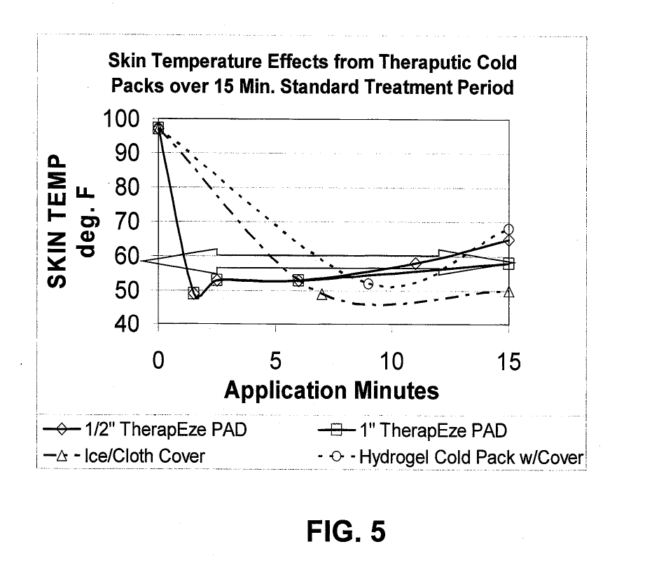

In cold therapy it is generally accepted that pain is reduced effectively at skin temperatures below 60° F. Skin temperatures were measured at timed intervals on patients undergoing testing with conventional ice packs, hydrogel pads and 2 sizes of pads of the present invention (pads of the present invention are labeled TherapEze in the graph of FIG. 5).

Results of cold therapy testing are as follows:
(a) In a typical 15 minute cold therapy treatment, it is evident from FIG. 5 that the present invention provides significantly faster therapeutic onset: 1 minute vs. 5 to 7 minutes.
(b) It is also evident that the time of effective therapy during a typical 15 minute treatment period is significantly greater than the conventional pads.
(c) Skin redness was noticed only on patients treated with ice.
(d) At about 3 minutes into the test, patients treated with the present invention reported a unique effect that sensation of the pad no longer felt cold even though infrared measurements of both the device and the skin underneath it demonstrated continued therapeutic efficacy and a possible anesthetic effect.

In exemplary embodiments where the thermal reservoir and skin contacting materials are filled silicone gel rubbers, these materials also offer superior durability, comfort, safety and therapeutic efficacy in heat therapy.

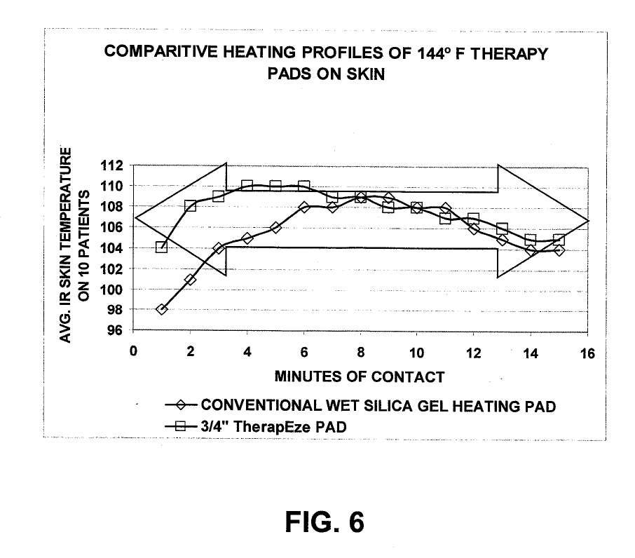

In heat therapy it is generally accepted that heat is therapeutic when skin temperatures are driven above 105° F. The present invention was tested as a heat therapy device with skin temperatures being measured at timed intervals on patients undergoing testing and compared to conventional hot wet sand or silica filled bags as used in physical therapy clinics (FIG. 6).

Results of heat testing are as follows:
(a) In a typical 15 minute heat therapy treatment, it is evident from FIG. 6 that the present invention provides significantly faster therapeutic onset: 1 minute vs. 3 minutes.
(b) Peak skin temperatures are slightly higher with the present invention than the conventional therapy pad, but in fact are reached significantly faster: 3 minutes vs. 8 minutes.
(c) Heated to 135° F., dry application of the present invention through a cotton undershirt is equally comfortable to the conventional pad at the same temperature applied through several layers of terrycloth.

In all examples tested with therapists and patients, a dominant comment was that the softness and flexibility of the present invention provided almost 100% contact on any part of the body treated. In contrast, the conventional heating and chilling pads often leave voids when bent around curvatures of the body.

Durability of the present invention is also remarkable when compared to gel bags that crack and split after a few freezing cycles or when compared to wet sand bags that perforate after two to three months use in a clinic.

### Application

This invention can be attached to a desired body part by hand holding, adhesive bonding or strapping, and is intended for direct contact with the skin in the cold therapy regime. The soft flexible nature of this invention allows for good conformation to body parts held with elastic straps.

In the cold therapy regime, the device is best activated by chilling to 32° F. or less with a freezer, a chemical chilling device or other method. The device is then mounted to the skin directly over a painful, swollen or inflamed area. As the contact shock is very brief, most people tolerate this invention placed on the skin as cold as −20° F.

In the hot therapy regime, the device is best activated by heating between 130° F. and 145° F. (according to patient comfort with a microwave oven, a hot water bath or other method). The device is then mounted dry to a thin cloth covering on the skin directly over a painful area. Typically, clothing such as an undershirt is adequate to maintain patient comfort.

For repeated use on multiple patients, cleaning and sterilization can be effected with detergents and water, alcohol and organic solvents.

The detailed description of exemplary embodiments of the invention herein shows various exemplary embodiments and the best modes, known to the inventor at this time, of the invention. These exemplary embodiments and modes are described in sufficient detail to enable those skilled in the art to practice the invention and are not intended to limit the scope, applicability, or configuration of the invention in any way. Rather, the following disclosure is intended to teach both the implementation of the exemplary embodiments and modes and any equivalent modes or embodiments that are known or obvious to those reasonably skilled in the art. Additionally, all included figures are non-limiting illustrations of the exemplary embodiments and modes, which similarly avail themselves to any equivalent modes or embodiments that are known or obvious to those reasonably skilled in the art.

Other combinations and/or modifications of structures, arrangements, applications, proportions, elements, materials, or components used in the practice of the instant invention, in addition to those not specifically recited, can be varied or otherwise particularly adapted to specific environments, manufacturing specifications, design parameters, or other operating requirements without departing from the scope of the instant invention and are intended to be included in this disclosure.

1. A solid hot and/or cold therapy device comprising:
a polymeric matrix with a physical property of low thermal conductivity;
a filler in the polymeric matrix with a physical property of high thermal conductivity.

2. The device of claim 1 wherein the thermal conductivity of the polymeric matrix is within a range of about 0.1 to 0.00001 Joules/cm sec. ° K.

3. The device of claim 1 wherein the thermal conductivity of the filler is within a range of about 0.1 to 4 Joules/cm sec. ° K.

4. The device of claim 1 wherein the specific heat of the filler is within a range of about 300 to 4500 Joules/Kg·° K.

5. The device of claim 1 wherein the specific heat of the polymer is within a range of about 300 to 4500 Joules/Kg·° K.

6. A hot and/or cold therapy device comprising:
a hollow elastomer body comprising a polymeric matrix with a physical property of low thermal conductivity and a filler in the polymeric matrix with a physical property of high thermal conductivity; and
an opening filled with at least one of a liquid, a solid and a gel.

7. The device of claim 6 wherein the thermal conductivity of the polymeric matrix is within a range of about 0.1 to 0.00001 Joules/cm sec. ° K.

8. The device of claim 6 wherein the thermal conductivity of the filler is within a range of about 0.1 to 4 Joules/cm sec. ° K.

9. The device of claim 6 wherein the specific heat of the filler is within a range of about 300 to 4500 Joules/Kg·° K.

10. The device of claim 6 wherein the specific heat of the polymer is within a range of about 300 to 4500 Joules/Kg·° K.

11. The device of claim 6 wherein said at least one of a liquid, a sold, and a gel has a high specific heat.

12. The device of claim 6 wherein the opening contains an active cooling and/or heating device.

13. A therapeutic contact layer having a thickness within a range of about 0.5 mm to 5 mm comprising:
a polymeric matrix with a physical property of low thermal conductivity;
a filler in the polymeric matrix with a physical property of high thermal conductivity.

14. The device of claim 13 wherein the thermal conductivity of the polymeric matrix is within a range of about 0.1 to 0.00001 Joules/cm sec. ° K.

15. The device of claim 13 wherein the thermal conductivity of the filler is within a range of about 0.1 to 4 Joules/cm sec. ° K.

16. The device of claim 13 wherein the specific heat of the filler is within a range of about 300 to 4500 Joules/Kg·° K.

17. The device of claim 13 wherein the specific heat of the polymeric matrix is within a range of about 300 to 4500 Joules/Kg·° K.

18. The device of claim 13 further comprising an active heating and/or cooling device attached to the therapeutic layer.
